(19) United States
(12) Patent Application Publication
(10) Pub. No.: US 2002/0016576 A1
(43) Pub. Date: Feb. 7, 2002

(54) Apparatus of eyedropper aiming

(76) Inventor: Wen-Tsao Lee, Hsinchu (TW)

Correspondence Address: WEN-TSAO LEE, NO. 10-17, SHIAUFENLIN, DALIN TSUEN, BEI PU, HSINCHU, 314, TW

(21) Appl. No.: 09/681,558

(22) Filed: Apr. 29, 2001

(30) Foreign Application Priority Data
Mar. 7, 2000 (TW) 089104043

(51) Int. Cl. A61M 35/00

(52) U.S. Cl. 604/300

(57) ABSTRACT

The Invention is an apparatus of eyedropper aiming which facilitates the user to adjust the position and angle of the eyedropper to drop the eye lotion accurately into the eye. A ray of light is projected and passes the eye lotion to the drop on the beak of the dropper. The light focuses on the drop and then is projected onto the user's eye. The user can easily see the light spot and adjust the position as well as the angle of the apparatus to have a clear round light spot will be imaged on the user's retina so that the user can drop the eye lotion accurately into the eye.

### BACKGROUND OF INVENTION

An Apparatus of Eyedropper Aiming

The Invention relates to an apparatus of eyedropper aiming with which a ray of light, penetrating the eye lotion, is focused and projected on the drop. By viewing the light spot on the drop, the user can adjust the position and angle of the apparatus to have a clear round light spot will be imaged on the user's retina so that the user can drop the eye lotion accurately into the eye.

While dropping eye lotion, the user usually gets nervous of being unable to see clearly the beak of the eyedropper as well as the drops. It is mainly because the shortest distance for human eyesight is about 25 cm. Due to the too short distance between the beak and the eyes, the user fails to judge the accurate position of the eyedropper only by his perception or experience. Therefore, the drops always fall outside of the eyes, causing waste of the eye lotion and reducing the medicinal virtues.

There is an eyedropper fixer, also an aiming design, which projects the image of the drop on the other eye with a convex lens. However, because human eyes can only judge the precise position of objects by meeting the 2 lines of sight, the single-eye aiming will cause considerable deviation. That is why so far there has not been an effective solution to the aiming problem.

The Invention is to improve the flaws of conventional eyedropper and provide an eyedropper aiming scheme which enables the user to adjust accurately the position along with the angle of the eyedropper by focusing the light on the drop and finally let the drop fall right into the user's eye.

### SUMMARY OF INVENTION

The concept of the Invention is based upon the theorems of the refraction on the liquid surface, the gravity that makes the drop always falling downward, and of the short focal distance of the drop. The apparatus of eyedropper aiming is affixed but removable at the bottom of the transparent eyedropper in consideration of convenient replacement of new eyedroppers and repeated use of the apparatus of eyedropper aiming. While pressing the circuit switch, the user simultaneously presses the eyedropper and a drop on the beak is formed. The incandescent lamp (IL) or the LED (light-emitting diode), once electrified, the light is focused by the focusing lens and projected to the surface of the lotion then through the eye lotion or the light-guide rode. The light is then focused on the drop and projected to the user's eye. By viewing the light, the user can adjust the position as well as angle of the eyedropper to let a clearer round light spot imaged on his retina, hence the drop will accurately fall into his or her eye.

The eye lotion, usually pellucid, has a permanent tendency to fall down vertically to the horizon by virtue of gravity. If the light projected by the apparatus of eyedropper aiming does not appear vertically, an angle will be created between the light path and the falling path of the drop, causing a failure for the drop to form a round light spot. Therefore, to ensure that the drop will make a focus on the end of the light path, the user has to operate the apparatus of eyedropper aiming vertically. By this way, a clear round light spot will be imaged on the user's retina after he or she sees in a short distance the light focused on the drop and thus enable the drop to fall accurately into the user's eye.

The lighting guidance design of this Invention offers a right solution to the problems of conventional eyedroppers. A transparent microphotograph can be equipped on the light path so when the apparatus of eyedropper aiming is positioned at the right angle, a clear picture image of the falling drop can be formed on the user's retina and helps the user easily locate the eyedropper, avoiding nervous blinks. Besides, a sound effect chip is implanted to the circuit of the apparatus of eyedropper aiming. By cooperating with a micro speaker, such as magnetic speaker or piezoelectric speaker, the sound effect chip can generate sound when the integrated circuit starts to function and thus reduce the user's pressure while dropping the eye lotion. Alternatively, a flashing LED or a flashing chip which provides flashing power source can be implanted to the circuit of the apparatus of eyedropper aiming so that the flashing light will distract the user's attention, avoid the nervous blinks and to ensure the lotion dropped accurately to the user's eye.

In consideration of size minimization, a mini mercury battery is applied into the apparatus of eyedropper aiming. Alternatively, a rechargeable battery such as Nickel-Cadmium or lithium battery can be used to further prolong the duration and reduce the cost. The rechargeable battery and the apparatus of eyedropper aiming are loaded together in a case, which offers electricity connection between the jack and the battery.

The characteristics of this Invention can be specifically presented by the following detailed figures.

### BRIEF DESCRIPTION OF DRAWINGS

FIG. 1 is a diagram illustration of operation for the falling of drop at right angle.

FIG. 2 is a diagram illustration of operation for the falling of drop at oblique angle.

FIG. 3 is a diagram illustration of the path of the light with a lens and a transparent microphotograph.

FIG. 4 is a diagram illustration of the path of the light with a lens a transparent microphotograph and a light-guide rode. [001 5] FIG. 5 is a front cross-sectional view and a side cross-sectional view of the first embodiment of the aiming apparatus.

FIG. 6 is a front sectional view of the first embodiment of the aiming apparatus and the eyedropper showing assembly and disassembly.

FIG. 7 is a front cross-sectional view and a side cross-sectional view of the second embodiment of the aiming apparatus.

FIG. 8 is an enlargement view of the light project unit and the beak.

FIG. 9 is front sectional views of the second embodiment of the aiming apparatus and a container with a light project unit showing the assembly and disassembly of the aiming apparatus.

FIG. 10 is a front cross-sectional view and a side cross-sectional view of the second embodiment of the aiming apparatus and a container with a light project unit.

FIG. 11 is an enlargement view of the beak of the light-guide rode.

FIG. 12 is cross-sectional views of the third embodiment of the aiming apparatus and a container with a light project unit and a light-guide rode showing the assembly and disassembly.

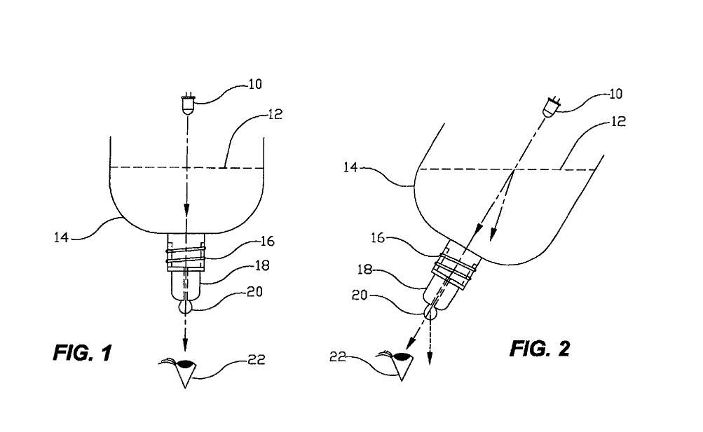

FIG. 1 3 is a front cross-sectional view and a side cross-sectional view of the third embodiment of the aiming apparatus and a container with light project unit and light-guide rode.

FIG. 14 is a front view and a side view of the second embodiment of the aiming apparatus and a container with a light project unit a break and a lid showing the assembly and disassembly.

FIG. 15 is a circuit diagram of the aiming apparatus.

FIG. 16 is a diagram of layout with an external battery.

### DETAILED DESCRIPTION

As shown in FIG. 1, the slant of the container 14 causes the light path and the surface of the liquid 12 of lotion at a non-vertical angle. When light source 10 is projected on the surface of the liquid 12 at a non-vertical angle, the refraction resulted from the shift of light from the air to the liquid of higher density will make the surface of the liquid 12 deviate from the light path. Besides, the direction of the gravity-influenced drop 20 is created at an angle with the light projected on the surface of the liquid 12. So when the additional light of the light path is projected aslant to the drop 20 on the beak 18 and focused on the drop 20, the user 22 can only receive an obscure non-round light spot on the retina. Therefore, the drop 20 will definitely falls outside of the user's 22 eye.

As shown in FIG. 2, the light source 10 is projected vertically onto the surface of the liquid 12 of the containers 4 and extends to the drop 20 on the beak 18. Influenced by the gravity, the drop 20 forms a focus on the light path. By observing in a short distance a round light spot which is created by the drop 20 on the beak 18, like a lens with a short focus, the user 22 receives a clear image on the retina hence helps the drop 20 accurately falls into the user's 22 eye.

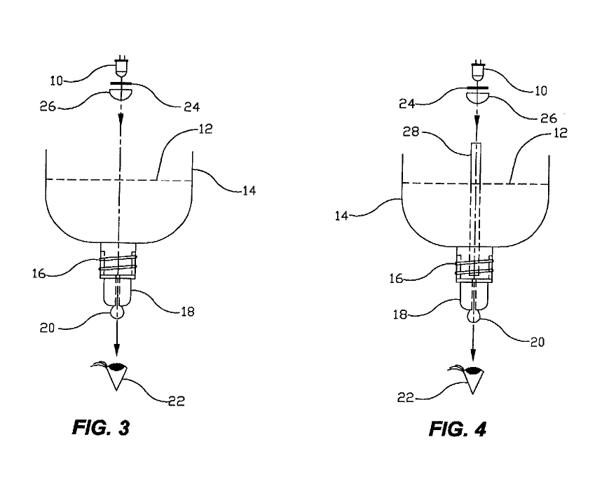

As shown in FIG. 3, the light source 10, penetrating the transparent microphotograph 24, is focused by the focusing lens 26 and then projected on the surface of the liquid 1 2 and the drop 20 on the beak 18. The light is further focused on the drop 20 and forms an image on the user's 22 retina.

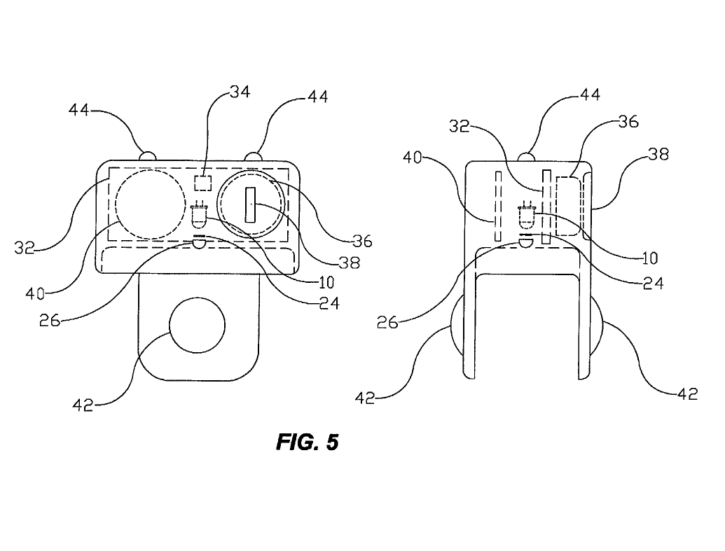

As shown in FIG. 4, if the eye lotion is made of liquid with less transparency, a light-guide rod 28, usually a round acrylic or PC plastic rod which is applied to connect the beak 1 8 and guide the proceeding of the light. When the light source 10 penetrates the transparent microphotograph 24 and is projected on the lightguide rode 28 after being focused on the focusing lens 26, the light can be guided by the light-guide rode 28 and projected directly to the drop 20 on the beak 18, regardless of the influence of the liquid of less transparency. The light can be focused on the drop 20 and finally forms an image on the user's 22 retina.

According to FIG. 5, a sound effect chip 34 is implanted to a printed circuit board 32 and integrated with a circuit of the light source 10, a battery 36, a sound generator 40, a dual power switch 42 and a pair of recharge terminal 44 in the shell of the housing. The recharge terminal 44 is utilized for the recharging purpose of the battery 36 with external battery 56. The battery cover 38 with whorls 16 can be unscrewed open for convenient battery replacement. The power switch 42 is a pressed-on film, which is equipped on the two wings of the apparatus of eyedropper aiming, extending to the center of the two wider sides of the container 14. While the user presses on the power switch 42, he presses the container 14 at the same time to let the drop 20 fall. The power switch 42 can be either a single or dual as needed.

At the bottom of the housing, there is a through hole, which the light source 10 can be projected with its light out vertically. The focusing lens 26 and the transparent microphotograph 24 are located on the light path between the through hole and the light source 10. The shell of bottom of the housing can be made to match with the shape of eyedroppers sold in the market.

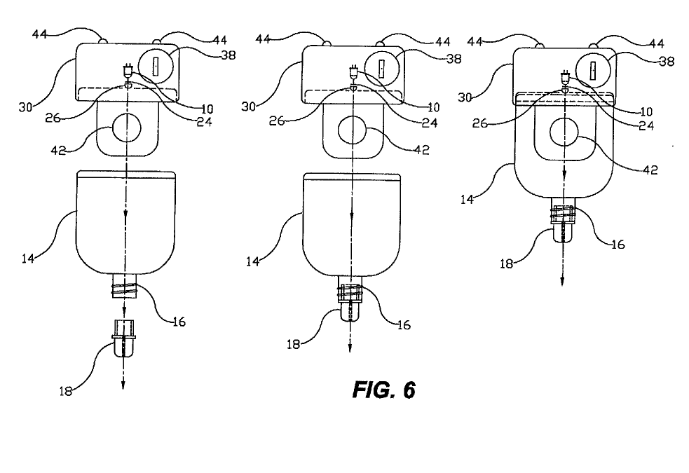

According to FIG. 6, when the aiming apparatus 30, the container 14 and the beak 18 are tightly fitted, the light from light source 10 on the aiming apparatus 30 is projected all the way through the microphotograph 24, the focusing lens 26, the hole at the bottom of the aiming apparatus 30, the transparent bottom of the container 14, the eye lotion and then to the beak 18, which creates a light path. A dual power switch 42 of the aiming apparatus 30 is located nearby the center of the two wider sides of the container 14.

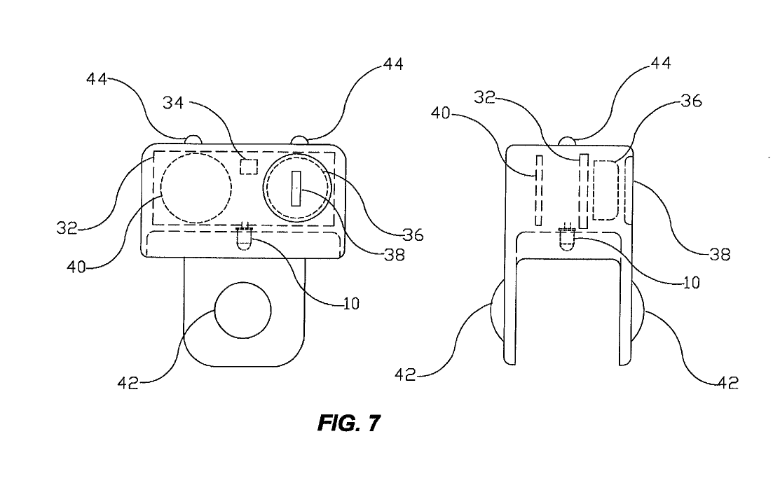

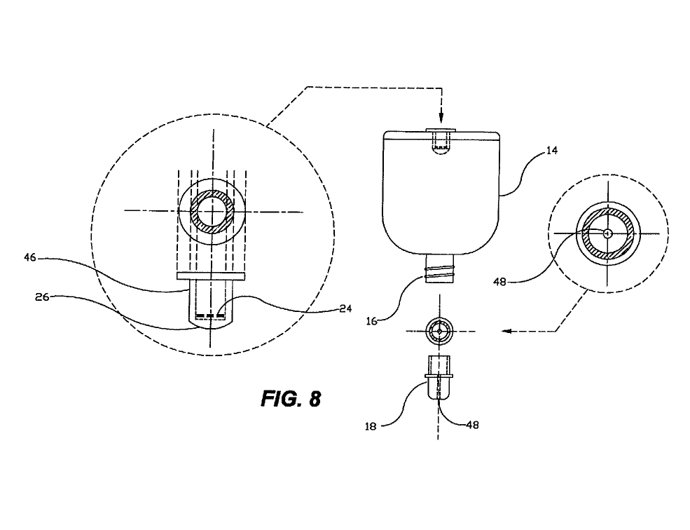

According to FIG. 7, a printed circuit board 32 with a sound effect chip 34 implanted, is integrated with a circuit of the light source 10, a battery 36, a sound generator 40, a dual power switch 42 and a pair of recharge terminal 44 within the housing of the aiming apparatus. The recharge terminal 44 is utilized for the recharging purpose of the battery 36 with the external battery 56. The battery cover 38 with whorls 16 can be unscrewed open for convenient battery replacement. The power switch 42 is a pressed-on film, which is equipped on the two wings of the aiming apparatus, extending to the center of the two wider sides of the container 14. While the user presses on the power switch 42, he presses the container 14 simultaneously to let the drop 20 fall. The power switch 42 can be either a single or dual as needed. At the bottom of the aiming apparatus, there is a hole where the light source is protruded.

According to FIG. 8, the light project unit 46 is a transparent plastic cap with fringe and in which the microphotograph 24 as well as the focusing lens 26 are installed. Alternatively the light project unit 46 can be united with the transparent plastic focusing lens 26. The light project unit 46 is located at the hole on the central bottom of the container 14 with glue or ultra sonic. The beak 18 with an aperture 48, which controls the flow of the eye lotion and makes the drops, is tightly fitted to the outlet of the container 14.

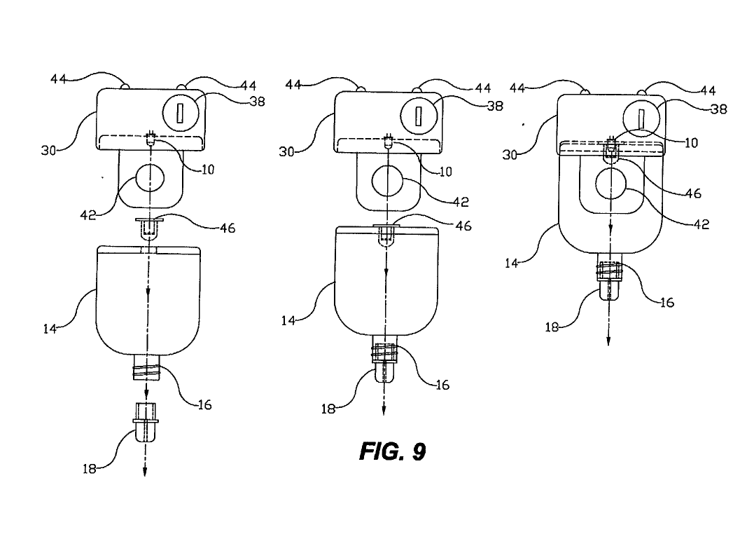

According to FIG. 9, the light from the light source 10 on the aiming apparatus 30 penetrates the light project unit 46 to the beak 18 and then creates a straight light path. When the aiming apparatus 30 is tightly fitted with the container 14 and the beak 18, the power switch 42 of the aiming apparatus 30, which is nearby the center of the container 14, can be easily started up while pressing the eye lotion out. The light source 10, which is protruded to the bottom of the aiming apparatus 30 is fitted into the light project unit 46 of the container 14.

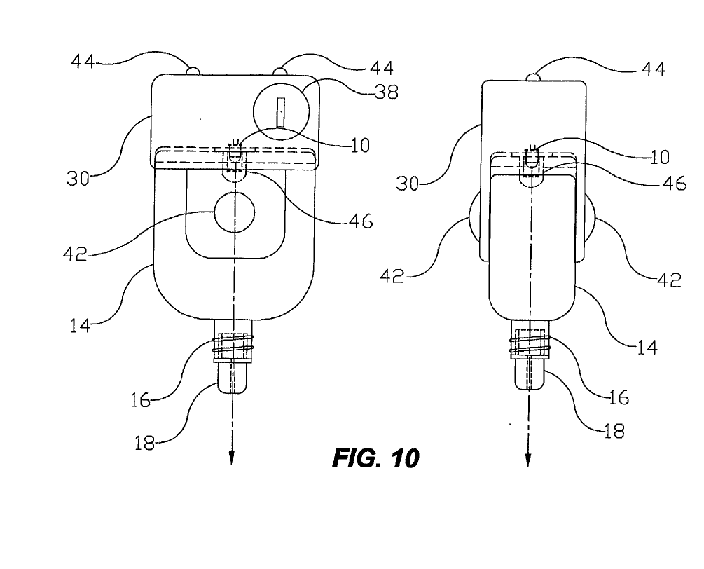

According to FIG. 10, when the aiming apparatus 30 is tightly fitted with the container 14 and the beak 18, the light from the light source 10, penetrating the light project unit 46 on the container 14 and being projected on the eye lotion and the beak 18, creates a straight light path. The dual power switch 42 of the aiming apparatus 30 is located nearby the center of the two wider sides of the container 14.

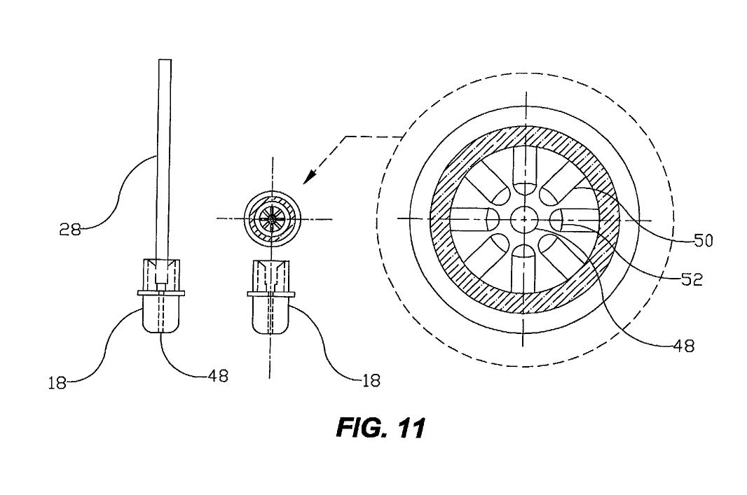

According to FIG. 11, in case that the eye lotion is made of liquid with less transparency, a light-guide rode 28 is fitted into the beak 18 to guide the light to the aperture 48. The light-guide hole 52 centered by the aperture 48 and forming a hole on the structure of multiple finny slices 50, which has a diameter shorter then that of the light-guide rode 28 so that the light-guide rode 28 can be tightly fitted into the light-guide hole 52. Furthermore, to make the eye lotion flow out smoothly, a gap is reserved between the light-guide hole 52 and the aperture 48.

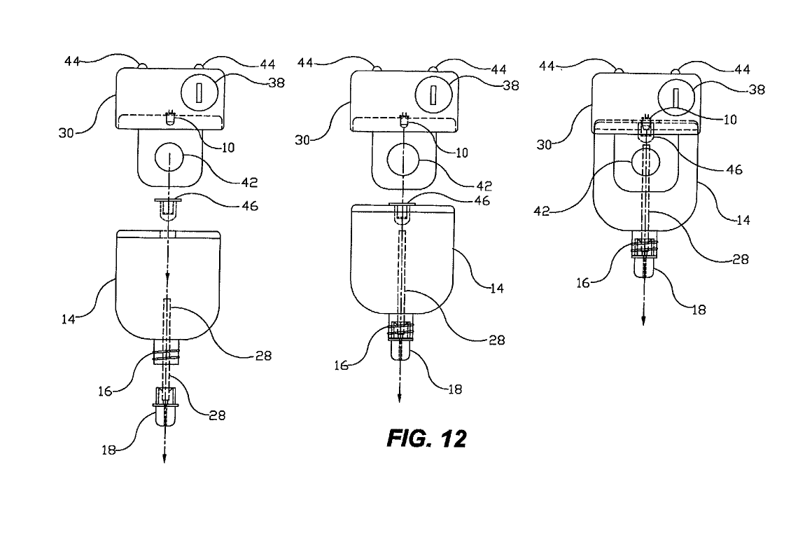

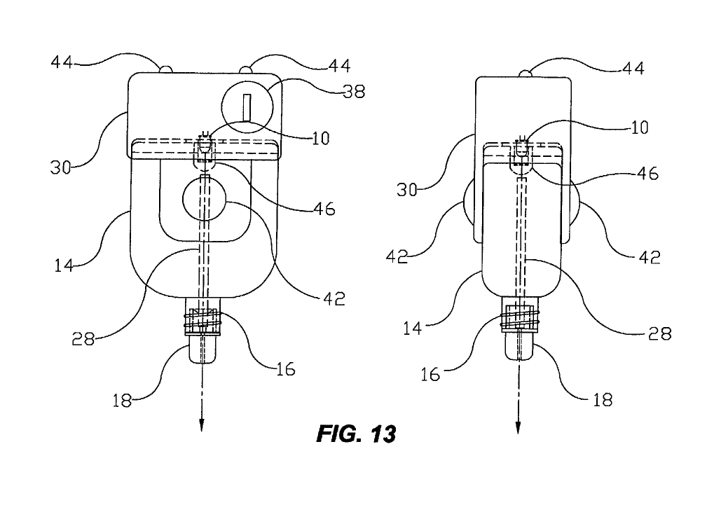

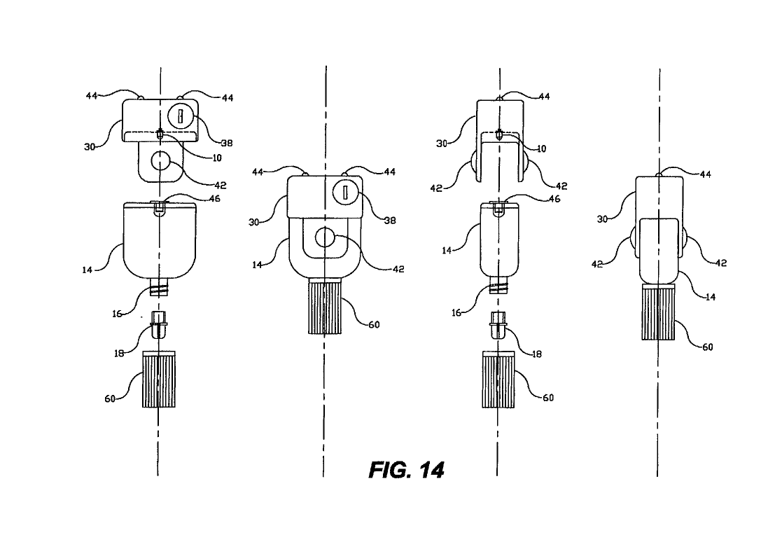

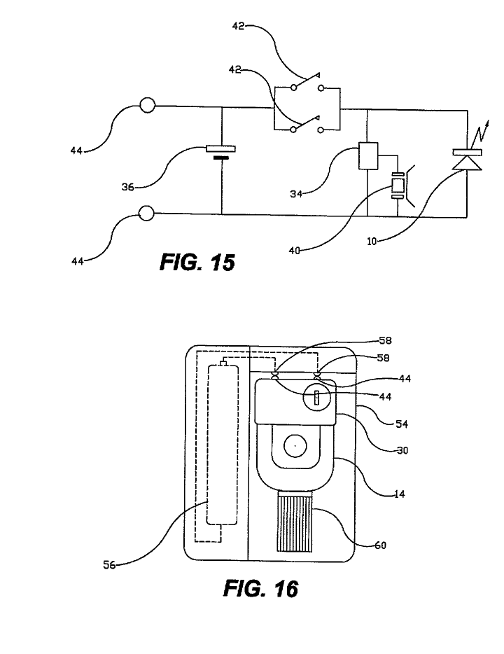

According to FIG. 12, the light from the light source 10 on the aiming apparatus 30 is projected to the light-guide rode 28. The light is guided by the light-guide rode 28 and extends to the beak 18 hence creates a light path. When the aiming apparatus 30 is tightly fitted with the container 14 and the beak 18, the power switch 42 of the aiming apparatus 30, which is nearby the center of the container 14 can be easily started up while pressing the eye lotion out. The light source 10, which is protruded to the bottom of the aiming apparatus 30 is fitted into the light project unit 46 of the container 14.

According to FIG. 13, when the light-guide rode 28, the beak 18, the aiming apparatus 30 and the container 14 are tightly fitted, a light from the light source 10 will penetrate the light project unit 46 and be projected to the light-guide rode 28 and then to the beak 18, thus a light path is created. The multiple power switches 42 of the aiming apparatus 30 is located nearby the center of the two wider sides of the container 14.

According to FIG. 14, The light project unit 46 is fitted into the center hole at the bottom of the container 14 and the beak 18 is fitted into the outlet of the container 14. The lid 60 of the container is screwed with the container 14 and the container 14 is fitted with the aiming apparatus 30. The power switch 42 of the aiming apparatus 30 is nearby the center of the container 14 and can be easily started up while pressing the eye lotion out. The light source 10, which is protruded to the bottom of the aiming apparatus 30 is fitted into the light project unit 46 of the container 14.

According to FIG. 1 5, a small mercury battery or alternatively a rechargeable lithium battery provides the power of the aiming apparatus. The power switch 42 can be either a single or dual circuit. The light source 1 0 can be an incandescent lamp (IL), or a LED (light-emitting diode), or a flashing LED. The integrated circuit of the sound effect chip 34 is connected with the lighting circuit. The dual recharge terminal 44 is equipped with rechargeable lithium battery, which can be charged by an external battery.

According to FIG. 16, the case 54 includes a bed for the external battery 56 whose dual recharge terminal 44 is connected to the dual power terminal 58 in the case 54. When the aiming apparatus 30, the container 14 and the lid 60 of the container are tightly fitted into the case 54, the multiple recharge terminal 44 and the dual power terminal 58 in the case 54 are connected and start to recharge the battery on the aiming apparatus 30.

It will be now apparent to those skilled in the art that other embodiments, detail and uses can be made consistent with the letter and spirit of the foregoing disclosure and within the scope of this patent, which is limited only by the following claims, construed in accordance with the patent law, including the doctrine of equivalents.

1. An apparatus of eyedropper aiming comprising:
a light source, which provides the light for aiming;
a battery, which provides the power for the light source;
a power switch, which controls the power on/off of the light source; and
a housing containing the light source, the battery and the power switch;
Wherein a light of the light source projected on the surface of the eye lotion and the beak, the light is then focused on the drop and further projected to the user's eye.

2. The apparatus of eyedropper aiming of claim 1 wherein said the light source is an incandescent lamp or a LED.

3. The apparatus of eyedropper aiming of claim 1 wherein further comprises a lens, which is located on the light path.

4. The apparatus of eyedropper aiming of claim 3 wherein furthermore comprises a transparent microphotograph, which is located on the light path between the light source and the lens.

5. The apparatus of eyedropper aiming of claim 1 wherein said the housing is fitted with an eyedropper.

6. The apparatus of eyedropper aiming of claim 1 wherein further comprising: an eye lotion container; a beak and a lid.

7. An apparatus of eyedropper aiming comprising:
a light source, which provides the light for aiming;
a battery, which provides the power for the light source;
a power switch, which controls the power on/off of the light source;
a housing containing the light source, the battery and the power switch;
an eye lotion container;
a light project unit is located on the light path of the container;
a beak where the eye lotion is pressed out; and
a lid, which stops the flow out of the eye lotion;
Wherein a light of the light source projected on the light project unit, to the surface of the eye lotion as well as the beak, the light is then focused on the drop and further projected to the user's eye.

8. The apparatus of eyedropper aiming of claim 7 wherein said the light source is an incandescent lamp or a LED.

9. The apparatus of eyedropper aiming of claim 2 or 8 wherein further comprises a flashing integrated circuit which provides flashing power source.

10. The apparatus of eyedropper aiming of claim 7 wherein further comprises a lens which is located on the light path of the light project unit.

11. The apparatus of eyedropper aiming of claim 10 wherein furthermore comprises a light-guide rode, which is located between the light project unit and the beak to guide the light on the light project unit to the beak.

12. The apparatus of eyedropper aiming of claim 11 wherein said the light-guide rode connected to the beak.

13. The apparatus of eyedropper aiming of claim 10 or 11 wherein furthermore comprises a transparent microphotograph, which is located on the light path between the light source and the lens.

14. The apparatus of eyedropper aiming of claim 1 or 7 wherein said the power switch is located nearby one side of the container.

15. The apparatus of eyedropper aiming of claim 1 or 7 wherein further comprises another power switch which is located nearby the other side of the container.

16. The apparatus of eyedropper aiming of claim 1 or 7 wherein said the light source is a flashing LED.

17. The apparatus of eyedropper aiming of claim 1 or 7 wherein further comprises a sound effect integrated circuit and a sound generator.

18. The apparatus of eyedropper aiming of claim 1 or 7 wherein said the battery is a rechargeable battery.

19. The apparatus of eyedropper aiming of claim 6 or 7 wherein said the container is fitted to the housing.
